(19) United States
(12) Patent Application Publication
(10) Pub. No.: US 2017/0014816 A1
(43) Pub. Date: Jan. 19, 2017

(54) PHOSPHORAMIDITE DERIVATIVES IN THE HYDROFORMYLATION OF OLEFIN-CONTAINING MIXTURES

(71) Applicants: Katrin Marie DYBALLA, Recklinghausen (DE); Robert FRANKE,, Marl (DE); Dirk FRIDAG, Haltern am See (DE); Eduard BENETSKIY, Ryazan, (RU); Armin BÖRNER, Rostock (DE); Detlef SELENT, Rostock (DE); EVONIK DEGUSSA GMBH, Essen (DE)

(72) Inventors: Katrin Marie DYBALLA, Recklinghausen (DE); Robert FRANKE, Marl (DE); Dirk FRIDAG, Haltern am See (DE); Eduard BENETSKIY, Ryazan (RU); Armin BÖRNER, Rostock (DE); Detlef SELENT, Rostock (DE)

(73) Assignee: EVONIK DEGUSSA GMBH, Essen (DE)

(21) Appl. No.: 14/906,696

(22) PCT Filed: Jul. 22, 2014
(86) PCT No.: PCT/EP2014/065722

§ 371 Date: Jan. 21, 2016

(30) Foreign Application Priority Data
Jul. 23, 2013 (DE) 10 2013 214 378.8

(51) Int. Cl. B01J 31/22; C07C 45/50; B01J 31/20; C07F 9/6571

(52) U.S. Cl. CPC B01J 31/2295; C07F 9/6571; C07C 45/505; B01J 31/20; B01J 31/2208; B01J 2231/321; B01J 2531/822; B01J 2531/004

(57) ABSTRACT

The invention relates to: a) phosphoramidites of formula (I); b) transition-metal-containing compounds of the formula Me(acac)(CO)L, wherein L is selected from formula (I); c) catalytically active compositions in hydroformylation that have the compounds mentioned under a) and b); d) a method for the hydroformylation of unsaturated compounds by using the catalytically active composition mentioned under c); and e) a multi-phase reaction mixture, containing unsaturated compounds, a gas mixture, which comprises carbon monoxide and hydrogen, aldehydes, and the catalytically active composition described under c).

In terms of volume, hydroformylation is one of the most important homogeneous catalyses on the industrial scale. The aldehydes obtained thereby are important intermediates or end products in the chemical industry (Rhodium Catalyzed Hydroformylation, P. W. N. M. van Leeuwen, C. Claver, eds.; Kluver Academic Publishers: Dordrecht Netherlands; 2000. R. Franke, D. Selent, A. Börner, Chem. Rev. 2012, 112, 5675). Hydroformylation with Rh catalysts is of particular significance.

For control of activity and regioselectivity of the catalyst, usually compounds of trivalent phosphorus are used as organic ligands. Particularly phosphites, i.e. compounds containing three P—O bonds, have become very widely used for this purpose (EP 0054986; EP 0697391; EP 213639; EP 214622; U.S. Pat. No. 4,769,498; DE 10031493; DE 102006058682; WO 2008124468).

Phosphoramidites, i.e. compounds having one or more P—N bonds rather than the P—O bonds, have to date been used only rarely as ligands in hydroformylation.

Van Leeuwen and coworkers (A. van Rooy, D. Burgers, P. C. J. Kamer, P. W. N. M. van Leeuwen, Recl. Trav. Chim. Pays-Bas 1996, 115, 492) were the first to study monodentate phosphoramidites in hydroformylation. Overall, only moderate catalytic properties were observed at the high to extremely high ligand/rhodium ratios of up to 1000:1. At the lowest ligand/rhodium ratio, or P/Rh ratio, of 10:1, a high isomerization activity and the formation of non-hydroformylated internal olefins was found. Only increasing the P/Rh ratio increased the TOF to a moderate 910 h⁻¹ and enhanced the selectivity.

The use of chiral phosphoramidites for asymmetric catalyses was claimed in WO 2007/031065, without giving working examples specifically for asymmetric hydroformylation. Chiral bidentate ligands each having a phosphoramidite unit have been used in various forms in asymmetric hydroformylation (J. Mazuela, O. Pàmies, M. Diéguez, L. Palais, S. Rosset, A. Alexakis, Tetrahedron: Asymmetry 2010, 21, 2153-2157; Y. Yan, X. Zhang, J. Am. Chem. Soc. 2006, 128, 7198-7202; Z. Hua, V. C. Vassar, H. Choi, I. Ojima, PNAS 2004, 13, 5411-5416).

Of paramount importance for the efficacy of the catalyst is the stability of the ligand towards various chemical agents before, during and after the catalysis (the latter in the case of intentional recycling). One of the main causes of the breakdown of phosphite ligands, which, unlike phosphines, are very stable towards oxygen, is the reaction with water, which leads to cleavage of the P—O bonds (Homogeneous Catalysts, Activity-Stability-Deactivation, P. W. N. M. van Leeuwen, J. C. Chadwick, eds.; Wiley-VCH, 2011, p. 23 ff.). The hydrolysis gives rise particularly to pentavalent phosphorus compounds which have lost most of their ligand properties. Water forms almost unavoidably under almost all hydroformylation conditions through aldol condensation of the product aldehydes.

In general, a greater tendency to react with nucleophiles is attributed to phosphoramidites than phosphites. This property is utilized widely, for example, for the synthesis of phosphites from phosphoramidites (e-EROS Encyclopedia of Reagents for Organic Synthesis. doi:10.1002/047084289X.rn00312; R. Hulst, N. K. de Vries, B. L. Feringa, Tetrahedron: Asymmetry 1994, 5, 699-708), but at the same time raises particular questions about the suitability thereof as ligands of long-term stability for catalysis.

The use of suitable phosphorus substituents can contribute to stabilization of phosphorus compounds at risk of hydrolysis. The only method described to date in the context of phosphoramidite ligands is the use of N-pyrrolyl radicals on the phosphorus (WO 02/083695). Substituents on the heterocycle, for example 2-ethylpyrrolyl (WO 03018192, DE 102005061642) or indolyl (WO 03/018192), improve hydrolysis stability still further.

The hydrolytic breakdown of phosphoramidite ligands can also be slowed by the addition of amines to the hydroformylation reaction, as taught in EP 1677911, US 2006/0224000 and U.S. Pat. No. 8,110,709.

The use of hydrolysis-stable pyrrolylphosphines or the addition of basic stabilizers greatly narrows the scope of application of the hydroformylation reaction to these working examples.

It is an object of the present invention to provide hydrolysis-stable ligands for catalytically active compositions for chemical synthesis of organic compounds, especially the hydroformylation, the hydrocyanation and the hydrogenation of unsaturated compounds. As well as the ease of synthesis of the phosphoramidites and the use thereof as ligands, a high yield of product and a high n/i selectivity are to be achieved in the hydroformylation.

The object is achieved by phosphoramidites of the formula (I):

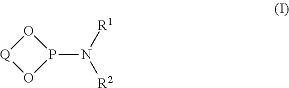

The present invention provides phosphoramidites of the formula (I) where Q is a divalent substituted or unsubstituted aromatic radical;

where R¹ is selected from alkyl, substituted or unsubstituted cycloalkyl and aryl radicals;

where R² is selected from alkyl, substituted or unsubstituted cycloalkyl and aryl radicals, but R¹ and R² are not i-propyl radicals, or R¹ and R² together with N form a heterocyclic structure.

In a particular embodiment, Q is selected from substituted or unsubstituted 1,1′-biphenyl, 1,1′-binaphthyl and ortho-phenyl radicals, especially substituted or unsubstituted 1,1′-biphenyl radicals.

In one variant of this embodiment, R¹ is selected from C₁-C₅-alkyl, substituted or unsubstituted cycloalkyl and aryl radicals; R² is selected from C₁-C₅-alkyl, substituted or unsubstituted cycloalkyl and aryl radicals, but R¹ and R² are not i-propyl radicals, or R¹ and R² together with N form a heterocyclic structure via alkylene groups.

In a further variant of this embodiment, R¹ is selected from C₁-C₅-alkyl, C₄-C₆-cycloalkyl and phenyl radicals; R² is selected from C₁-C₅-alkyl, C₄-C₆-cycloalkyl and phenyl radicals, but R¹ and R² are not i-propyl radicals, or R¹ and R² together with N form a heterocyclic structure via alkylene groups.

In a particularly preferred embodiment, the compounds of the formula (I) are selected from:

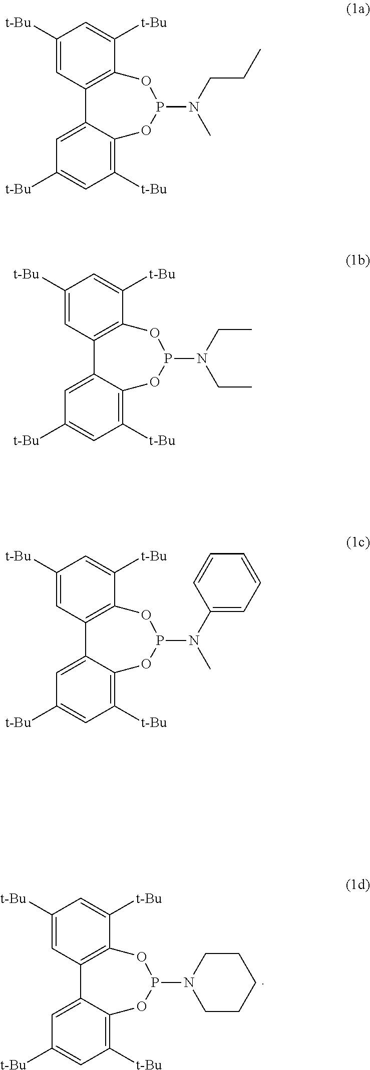 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

The present invention further provides transition metal compounds of the formula Me(acac)(CO)L with Me=transition metal, where L is selected from:

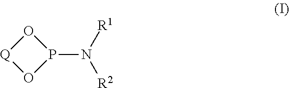

where Q is a divalent substituted or unsubstituted aromatic radical;

where R¹ is selected from alkyl, substituted or unsubstituted cycloalkyl and aryl radicals;

where R² is selected from alkyl, substituted or unsubstituted cycloalkyl and aryl radicals, but R¹ and R² are not i-propyl radicals, or R¹ and R² together with N form a heterocyclic structure.

In a particular embodiment, Q is selected from substituted or unsubstituted 1,1′-biphenyl, 1,1′-binaphthyl and ortho-phenyl radicals, especially substituted or unsubstituted 1,1′-biphenyl radicals. In one variant of this embodiment, R¹ is selected from C₁-C₅-alkyl, substituted or unsubstituted cycloalkyl and aryl radicals; R² is selected from C₁-C₅-alkyl, substituted or unsubstituted cycloalkyl and aryl radicals, but R¹ and R² are not i-propyl radicals, or R¹ and R² together with N form a heterocyclic structure via alkylene groups.

In a further variant of this embodiment, R¹ is selected from C₁-C₅-alkyl, C₄-C₆-cycloalkyl and phenyl radicals; R² is selected from C₁-C₅-alkyl, C₄-C₆-cycloalkyl and phenyl radicals, but R¹ and R² are not i-propyl radicals, or R¹ and R² together with N form a heterocyclic structure via alkylene groups.

In a particularly preferred embodiment, the compounds of the formula (I) are selected from:

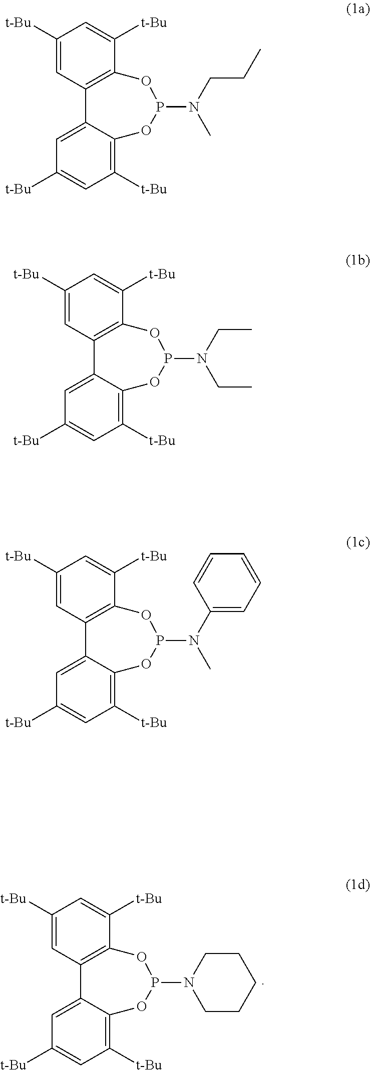 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

In a particularly preferred embodiment, the transition metal Me is selected here from ruthenium, cobalt, rhodium, iridium; especially preferably, Me=rhodium.

The transition metal is contacted with the inventive phosphoramidites as a precursor in the form of its salts, for example the halides, carboxylates—e.g. acetates—or commercially available complexes, for example acetylacetonates, carbonyls, cyclopolyenes—e.g. 1,5-cyclooctadiene—or else mixed forms thereof, for example Rh(acac)(CO)₂ with acac=acetylacetonate anion, Rh(acac)(COD) with COD=1,5-cyclooctadiene, and this reaction can be effected in a preceding reaction or else in the presence of a hydrogen- and carbon monoxide-containing gas mixture.

The present invention also provides catalytically active compositions in the hydroformylation comprising:

a) transition metal compounds of the formula Me(acac)(CO)L with Me=transition metal, where L is selected from:

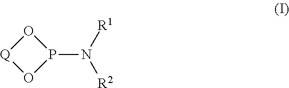

where Q is a divalent substituted or unsubstituted aromatic radical;

where R¹ is selected from alkyl, substituted or unsubstituted cycloalkyl and aryl radicals;

where R² is selected from alkyl, substituted or unsubstituted cycloalkyl and aryl radicals, but R¹ and R² are not i-propyl radicals, or R¹ and R² together with N form a heterocyclic structure.

b) free ligands of the formula (I):

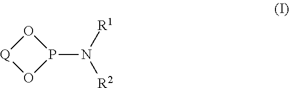

where Q is a divalent substituted or unsubstituted aromatic radical;

where R¹ is selected from alkyl, substituted or unsubstituted cycloalkyl and aryl radicals;

where R² is selected from alkyl, substituted or unsubstituted cycloalkyl and aryl radicals, but R¹ and R² are not i-propyl radicals, or R¹ and R² together with N form a heterocyclic structure.

c) solvents.

In the context of the present invention, solvents are regarded as being not only substances that have no inhibiting effect on product formation—having been added externally to the reaction mixture or initially charged therein—but also mixtures of compounds which form from side reactions or further reactions of the products in situ; for example what are called high boilers which form from the aldol condensation, the acetalization of the primary aldehyde product or else esterification, and lead to the corresponding aldol products, formates, acetals and ethers. Solvents initially charged externally in the reaction mixture may be aromatics, for example toluene-rich aromatics mixtures, or alkanes or mixtures of alkanes.

In general, high boilers are understood to mean those substances or else substance mixtures that boil at a higher temperature than the primary aldehyde product and have higher molar masses than the primary aldehyde product.

In a particular embodiment of the compositions that are catalytically active in the hydroformylation, the structural element Q—both in the transition metal compounds and in the free ligands—is selected from substituted or unsubstituted 1,1′-biphenyl, 1,1′-binaphthyl and ortho-phenyl radicals, especially substituted or unsubstituted 1,1′-biphenyl radicals.

In one variant of this embodiment, R¹ is selected from C₁-C₅-alkyl, substituted or unsubstituted cycloalkyl and aryl radicals; R² is selected from C₁-C₅-alkyl, substituted or unsubstituted cycloalkyl and aryl radicals, but R¹ and R² are not i-propyl radicals, or R¹ and R² together with N form a heterocyclic structure via alkylene groups.

In a further variant of this embodiment, R¹ is selected from C₁-C₅-alkyl, C₄-C₆-cycloalkyl and phenyl radicals; R² is selected from C₁-C₅-alkyl, C₄-C₆-cycloalkyl and phenyl radicals, but R¹ and R² are not i-propyl radicals, or R¹ and R² together with N form a heterocyclic structure via alkylene groups.

In a particular embodiment, the transition metal compounds are of the formula Me(acac)(CO)L with Me=transition metal, where L is selected from:

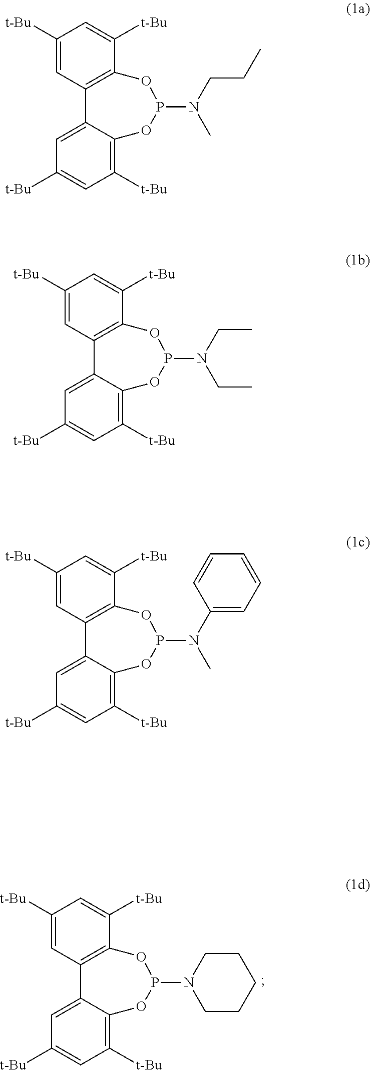 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

the free ligands are selected from:

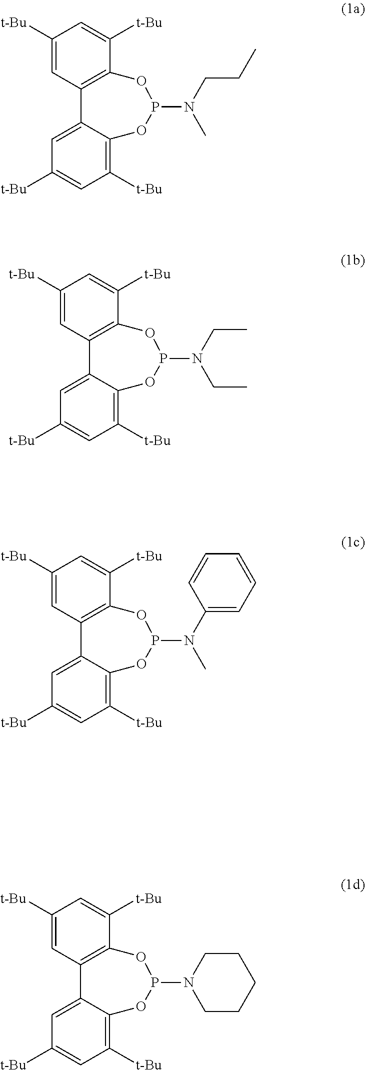 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

In a particularly preferred embodiment, the transition metal Me is selected here from ruthenium, cobalt, rhodium, iridium; especially preferably, Me=rhodium.

The present invention further provides:

for the use of the catalytically active compositions in a process for hydroformylating unsaturated compounds and

a process for hydroformylating unsaturated compounds using said catalytically active composition, where the unsaturated compounds are selected from:
hydrocarbon mixtures from steamcracking plants;
hydrocarbon mixtures from catalytically operated cracking plants;
hydrocarbon mixtures from oligomerization processes;
hydrocarbon mixtures comprising polyunsaturated compounds;
olefin-containing mixtures including olefins having up to 30 carbon atoms.

The unsaturated compounds which are hydroformylated in the process according to the invention include hydrocarbon mixtures obtained in petrochemical processing plants. Examples of these include what are called C₄ cuts. Typical compositions of C₄ cuts from which the majority of the polyunsaturated hydrocarbons has been removed and which can be used in the process according to the invention are listed in Table 1 below (see DE 10 2008 002188).

TABLE 1

| | Steamcracking | | Steamcracking | | Catalytic | |
|---|---|---|---|---|---|---|
| | plant | | plant | | cracking plant | |
| Component | HCC₄ | HCC₄/SHP | Raff. I | Raff. I/SHP | CC₄ | CC₄/SHP |
| isobutane | 1-4.5 | 1-4.5 | 1.5-8 | 1.5-8 | 37 | 37 |
| [% by | | | | | | |
| mass] | | | | | | |
| n-butane | 5-8 | 5-8 | 6-15 | 6-15 | 13 | 13 |
| [% by | | | | | | |
| mass] | | | | | | |
| E-2-butene | 18-21 | 18-21 | 7-10 | 7-10 | 12 | 12 |
| [% by | | | | | | |
| mass] | | | | | | |
| 1-butene | 35-45 | 35-45 | 15-35 | 15-35 | 12 | 12 |
| [% by | | | | | | |
| mass] | | | | | | |
| isobutene | 22-28 | 22-28 | 33-50 | 33-50 | 15 | 15 |
| [% by | | | | | | |
| mass] | | | | | | |
| Z-2-butene | 5-9 | 5-9 | 4-8 | 4-8 | 11 | 11 |
| [% by | | | | | | |
| mass] | | | | | | |
| 1,3- | 500-8000 | 0-50 | 50-8000 | 0-50 | <10000 | 0-50 |
| butadiene | | | | | | |
| [ppm by | | | | | | |
| mass] | | | | | | |

Key:
HCC₄: typical of a C₄ mixture which is obtained from the C₄ cut from a steamcracking plant (high severity) after the hydrogenation of the 1,3-butadiene without additional moderation of the catalyst.
HCC₄/SHP: HCC₄ composition in which residues of 1,3-butadiene have been reduced further in a selective hydrogenation process/SHP.
Raff. I (raffinate I): typical of a C₄ mixture which is obtained from the C₄ cut from a steamcracking plant (high severity) after the removal of the 1,3-butadiene, for example by an NMP extractive rectification.
Raff. I/SHP: raff. I composition in which residues of 1,3-butadiene have been reduced further in a selective hydrogenation process/SHP.
CC₄: typical composition of a C₄ cut which is obtained from a catalytic cracking plant.
CC₄/SHP: composition of a C₄ cut in which residues of 1,3-butadiene have been reduced further in a selective hydrogenation process/SHP.

In one variant of the process, the unsaturated compound or mixture thereof has been selected from:
hydrocarbon mixtures from steamcracking plants;
hydrocarbon mixtures from catalytically operated cracking plants, for example FCC cracking plants;
hydrocarbon mixtures from oligomerization processes in the homogeneous phase and heterogeneous phases, for example the OCTOL, DIMERSOL, Fischer-Tropsch, Polygas, CatPoly, InAlk, Polynaphtha, Selectopol, MOGD, COD, EMOGAS, NExOCTANE or SHOP process;
hydrocarbon mixtures comprising polyunsaturated compounds.

In one variant of the process, the mixture includes unsaturated compounds having 2 to 30 carbon atoms.

In one variant of the process, the mixture includes unsaturated compounds having 2 to 8 carbon atoms.

In a further variant of the process, the mixture includes polyunsaturated hydrocarbons.

In a particular embodiment, the mixture comprises butadienes.

In particularly preferred embodiments of the process according to the invention, olefin-containing mixtures hydroformylated are n-octenes, 1-octene and C₈-containing olefin mixtures.

In a further embodiment of the process according to the invention, in a first process step, phosphoramidites of the formulae (I):

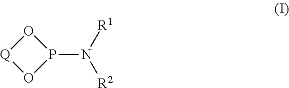

where Q is a divalent substituted or unsubstituted aromatic radical;

where R¹ is selected from alkyl, substituted or unsubstituted cycloalkyl and aryl radicals;

where R² is selected from alkyl, substituted or unsubstituted cycloalkyl and aryl radicals, but R¹ and R² are not i-propyl radicals, or R¹ and R² together with N form a heterocyclic structure;

in a particular embodiment, Q is selected from substituted or unsubstituted 1,1′-biphenyl, 1,1′-binaphthyl and ortho-phenyl radicals, especially substituted or unsubstituted 1,1′-biphenyl radicals;

in one variant of this embodiment, R¹ is selected from C₁-C₅-alkyl, substituted or unsubstituted cycloalkyl and aryl radicals; R² is selected from C₁-C₅-alkyl, substituted or unsubstituted cycloalkyl and aryl radicals, but R¹ and R² are not i-propyl radicals, or R¹ and R² together with N form a heterocyclic structure via alkylene groups;

in a further variant of this embodiment, R¹ is selected from C₁-C₅-alkyl, C₄-C₆-cycloalkyl and phenyl radicals; R² is selected from C₁-C₅-alkyl, C₄-C₆-cycloalkyl and phenyl radicals, but R¹ and R² are not i-propyl radicals, or R¹ and R² together with N form a heterocyclic structure via alkylene groups;

especially of the formulae:

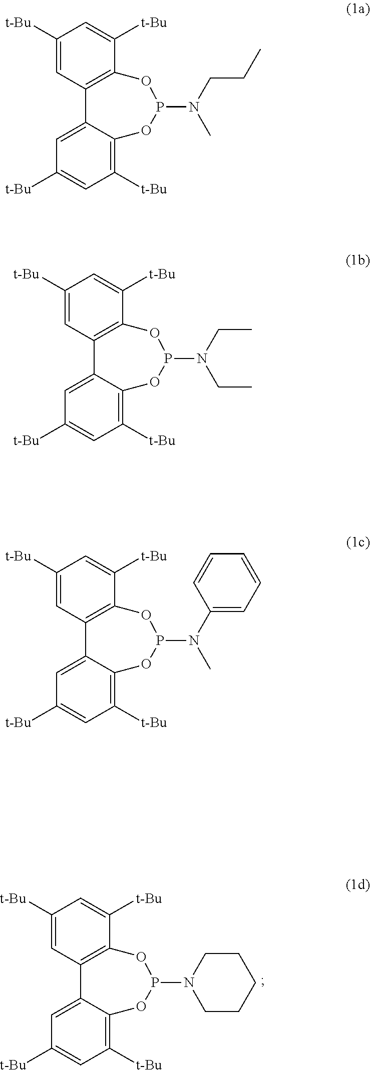 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

are initially charged as ligands in at least one reaction zone, and reacted with a precursor of the transition metal to give a transition metal compound of the formula Me(acac)(CO)L where L is selected from:

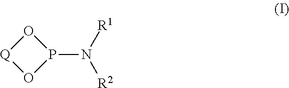

where Q is a divalent substituted or unsubstituted aromatic radical;

where R¹ is selected from alkyl, substituted or unsubstituted cycloalkyl and aryl radicals;

where R² is selected from alkyl, substituted or unsubstituted cycloalkyl and aryl radicals, but R¹ and R² are not i-propyl radicals, or R¹ and R² together with N form a heterocyclic structure;

in a particular embodiment, Q is selected from substituted or unsubstituted 1,1′-biphenyl, 1,1′-binaphthyl and ortho-phenyl radicals, especially substituted or unsubstituted 1,1′-biphenyl radicals;

in one variant of this embodiment, R¹ is selected from C₁-C₅-alkyl, substituted or unsubstituted cycloalkyl and aryl radicals; R² is selected from C₁-C₅-alkyl, substituted or unsubstituted cycloalkyl and aryl radicals, but R¹ and R² are not i-propyl radicals, or R¹ and R² together with N form a heterocyclic structure via alkylene groups;

in a further variant of this embodiment, R¹ is selected from C₁-C₅-alkyl, C₄-C₅-cycloalkyl and phenyl radicals; R² is selected from C₁-C₅-alkyl, C₄-C₆-cycloalkyl and phenyl radicals, but R¹ and R² are not i-propyl radicals, or R¹ and R² together with N form a heterocyclic structure via alkylene groups;

especially of the formulae:

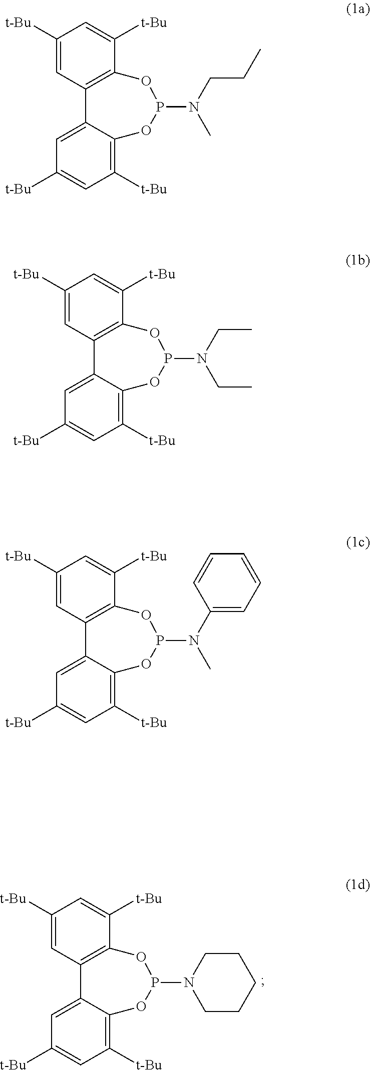 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

and finally, after adding free ligands of the formula (I):

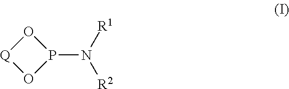

where Q is a divalent substituted or unsubstituted aromatic radical;

where R¹ is selected from alkyl, substituted or unsubstituted cycloalkyl and aryl radicals;

where R² is selected from alkyl, substituted or unsubstituted cycloalkyl and aryl radicals, but R¹ and R² are not i-propyl radicals, or R¹ and R² together with N form a heterocyclic structure;

in a particular embodiment, Q is selected from substituted or unsubstituted 1,1′-biphenyl, 1,1′-binaphthyl and ortho-phenyl radicals, especially substituted or unsubstituted 1,1′-biphenyl radicals;

in one variant of this embodiment, R¹ is selected from C₁-C₅-alkyl, substituted or unsubstituted cycloalkyl and aryl radicals; R² is selected from C₁-C₅-alkyl, substituted or unsubstituted cycloalkyl and aryl radicals, but R¹ and R² are not i-propyl radicals, or R¹ and R² together with N form a heterocyclic structure via alkylene groups;

in a further variant of this embodiment, R¹ is selected from C₁-C₅-alkyl, C₄-C₆-cycloalkyl and phenyl radicals; R² is selected from C₁-C₅-alkyl, C₄-C₆-cycloalkyl and phenyl radicals, but R¹ and R² are not i-propyl radicals, or R¹ and R² together with N form a heterocyclic structure via alkylene groups;

especially of the formulae:

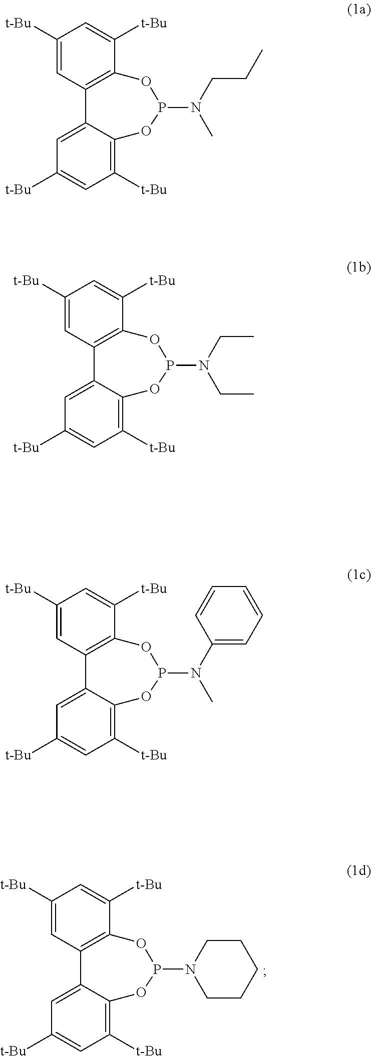 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

and also solvents and a carbon monoxide- and hydrogen-containing gas mixture, to give a catalytically active composition in the hydroformylation;

in a subsequent step, the unsaturated compounds are added under the reaction conditions to form a polyphasic reaction mixture;

after the end of the reaction, the reaction mixture is separated into aldehydes, alcohols, high boilers, ligands, degradation products of the catalytically active composition.

In a further embodiment of the process according to the invention, the unsaturated compounds are added together with the the precursor of the transition metal and the ligands, preferably when the unsaturated compounds are in a liquid state of matter at room temperature and standard pressure corresponding to 1013 hPa.

In the context of this invention, degradation products are regarded as being substances which originate from the breakdown of the composition catalytically active in the hydroformylation. They are described, for example, in U.S. Pat. No. 5,364,950, U.S. Pat. No. 5,763,677, and also in Catalyst Separation, Recovery and Recycling, edited by D. J. Cole-Hamilton, R. P. Tooze, 2006, NL, pages 25-26, and in Rhodium-catalyzed Hydroformylation, ed. by P. W. N. M. van Leeuwen et C. Clever, Kluwer Academic Publishers 2006, AA Dordrecht, NL, pages 206-211.

The present invention finally provides a polyphasic reaction mixture comprising:
unsaturated compounds;
a gas mixture including carbon monoxide, hydrogen;
catalytically active compositions comprising:

a) transition metal compounds of the formula Me(acac)(CO)L with Me=transition metal, where L is selected from:

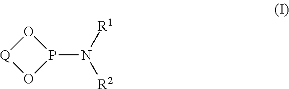

b) free ligands of the formulae (I):

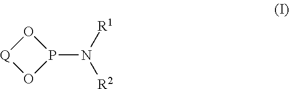

c) solvents.

In a particular embodiment, the polyphasic reaction mixture includes the transition metal compounds of the formula Me(acac)(CO)L with Me=transition metal, where L is selected from:

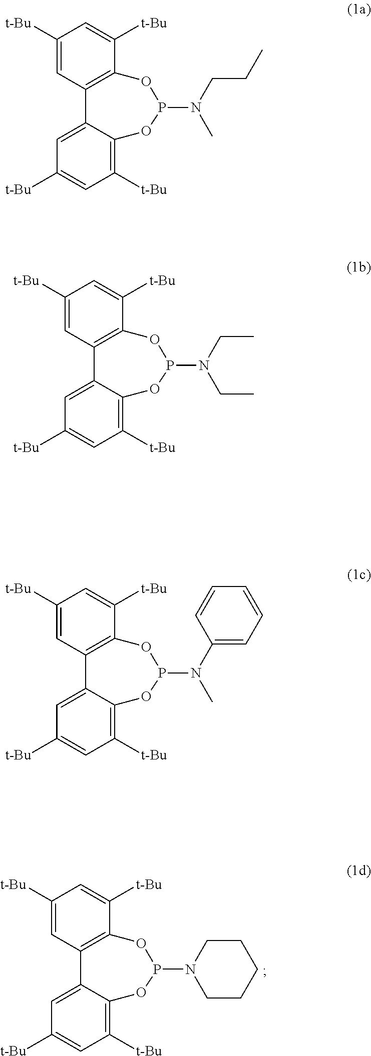 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

where the free ligands are selected from:

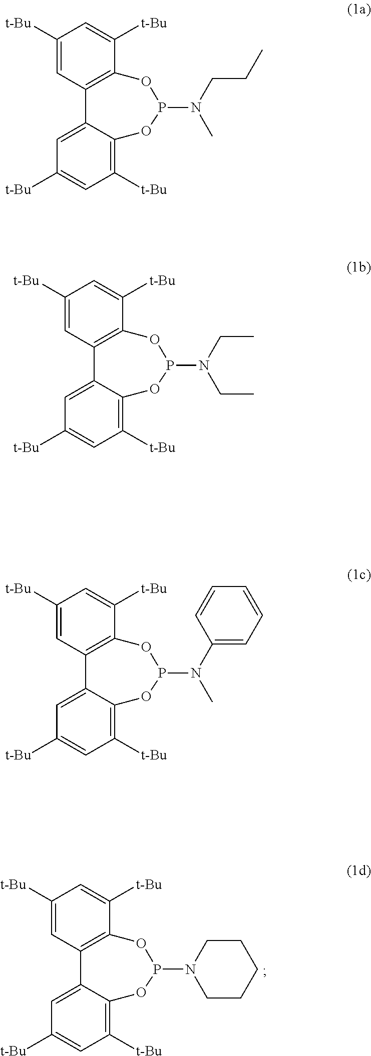 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

where the transition metal Me is selected from ruthenium, cobalt, rhodium, iridium, especially rhodium;

where the unsaturated compounds are selected from:
hydrocarbon mixtures from steamcracking plants;
hydrocarbon mixtures from catalytically operated cracking plants, for example FCC cracking plants;
hydrocarbon mixtures from oligomerization processes in the homogeneous phase and heterogeneous phases, for example the OCTOL, DIMERSOL, Fischer-Tropsch, Polygas, CatPoly, InAlk, Polynaphtha, Selectopol, MOGD, COD, EMOGAS, NExOCTANE or SHOP process;
hydrocarbon mixtures comprising polyunsaturated compounds;

where the solvent is added externally and does not intervene in an inhibiting fashion in the hydroformylation reaction, especially when the solvent is formed in situ from the primary products.

### EXAMPLES

General Working Methods

All the preparations which follow were conducted with standard Schlenk technology under protective gas. The solvents were dried over suitable desiccants before use (Purification of Laboratory Chemicals, W. L. F. Armarego (Author), Christina Chai (Author), Butterworth Heinemann (Elsevier). 6th edition, Oxford 2009).

Phosphorus trichloride (Aldrich) was distilled under argon before use. All preparative operations were effected in baked-out vessels. The products were characterized by means of NMR spectroscopy. Chemical shifts are reported in ppm. The ³¹P NMR signals were referenced according to: SR_{31P}═SR_{1H}*(BF_{31P}/BF_{1H})═SR_{1H}*0.4048. (Robin K. Harris, Edwin D. Becker, Sonia M. Cabral de Menezes, Robin Goodfellow, and Pierre Granger, Pure Appl. Chem., 2001, 73, 1795-1818; Robin K. Harris, Edwin D. Becker, Sonia M. Cabral de Menezes, Pierre Granger, Roy E. Hoffman and Kurt W. Zilm, Pure Appl. Chem., 2008, 80, 59-84).

The recording of nuclear resonance spectra was effected on Bruker Avance 300 or Bruker Avance 400, gas chromatography analysis on Agilent GC 7890A, elemental analysis on Leco TruSpec CHNS and Varian ICP-OES 715, and ESI-TOF mass spectrometry on Thermo Electron Finnigan MAT 95-XP and Agilent 6890 N/5973 instruments,

### Example 1

General Synthesis Method.

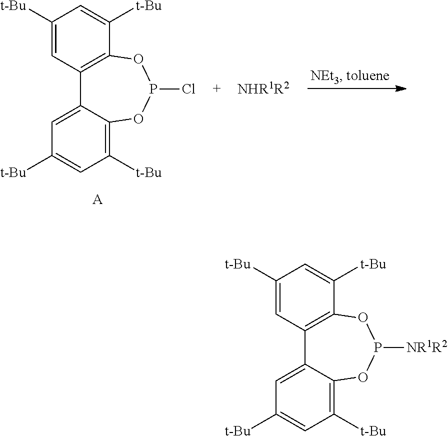

To a stirred solution of the chlorophosphite A (4 mmol) (preparation according to US 20080188686 A1) in toluene (15 ml) were added Et₃N (8 mmol) and the appropriate amine (4.8 mmol). The solution was stirred at room temperature. The progress of the reaction was monitored by means of ³¹P NMR spectroscopy. Once the chlorophosphite had been fully converted (2-10 h), the readily evaporable liquids were distilled off under reduced pressure. Subsequently, dried toluene (15 ml) was again added. The resultant suspension was filtered through a layer of neutral alumina (about 3 cm, Ø=2 cm; Schlenk filter, porosity 4) and then washed through with toluene (10 ml). After the solution had been concentrated, the residue was dried under reduced pressure at 45-50° C. for 3 h. If necessary, the product can be purified by recrystallization.

### Example 2

### N-(2,4,8,10-Tetra-tert-butyldibenz[d,f]{1,3,2}dioxaphosphepin-6-yl)-N-methylpropylamine

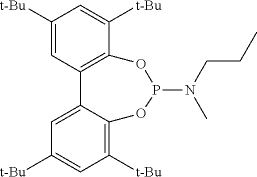 [SMILES: CCCN(C)p1oc2c(C(C)(C)C)cc(C(C)(C)C)cc2c2cc(C(C)(C)C)cc(C(C)(C)C)c2o1]

(1a)

The compound was prepared analogously to the method of Example 1. Yield: 89%; white solid. ¹H NMR (300 MHz, CDCl₃): δ 0.76 (br. s, 3H), 1.27 (s, 18H), 1.39-1.41 (2× overlapping signals, 18H+2H), 2.28 (br, s, 3H), 2.91 (br, s, 2H), 7.09 (d, 2H, J=2.5 Hz), 7.32 (d, 2H, J=2.5 Hz). ³¹P NMR (121 MHz, CDCl₃): δ 147.8 (br. s). ¹³C NMR (75 MHz, CDCl₃): δ 11.1 (s, CH₃CH₂), 21.0 (d, ³J=3.7 Hz, CH₃CH₂), 30.9 (d, J=2.8 Hz, (CH₃)₃C), 31.6 (s, (CH₃)₃C), 34.6 (s, (CH₃)₃C), 35.4 (s, (CH₃)₃C), 51.2 (br, s, NCH₂), 123.9 (s, CH_{Ar}), 126.2 (s, CH_{Ar}), 132.7 (d, J=3.6 Hz, C_{Ar}), 139.7 (s, C_{Ar}), 145.6 (s, C_{Ar}), 147.8 (d, J=5.4 Hz, C_{Ar}).MS (EI, 70 eV): m/z (I, %): 511 (10), 439 (39), 72 (9), 57 (100). HRMS (ESI-TOF/MS): calculated: m/z (C₃₂H₅₁N₁O₂P₁, (M+H)⁺) 512.36519; found 512.36557; calculated m/z (C₃₂H₅₀N₁Na₁O₂P₁, (M+Na)⁺) 534.34714; found 534.34778. Anal. calculated for C₃₂H₅₀N₁O₂P₁: C, 75.11; H, 9.85; N, 2.74; P, 6.05. Found: C, 74.64; H, 10.19; N, 2.40; P, 5.95.

The compound was prepared analogously to the method of Example 1. Yield: 98%; white solid; ¹H NMR (300 MHz, CDCl₃): δ 0.72 (t, 3H, J=7.4 Hz), 1.26 (s, 18H), 1.36-1.38 (2× overlapping singlets, 20H), 2.67 (pentet, 2H, J=7.4 Hz), 2.84-3.00 (m, 1H), 7.07 (d, 2H, J=2.4 Hz), 7.32 (d, 2H, J=2.4 Hz). ³¹P NMR (121 MHz, CDCl₃): δ 148.0 (s). ¹³C NMR (62 MHz, CDCl₃): δ 11.1 (s, CH₃), 26.0 (d, J=3.4 Hz, CH₂), 31.2 (d, J=2.8 Hz, (CH₃)₃C), 31.6 (s, (CH₃)₃C), 34.6 (s, (CH₃)₃C), 35.6 (s, (CH₃)₃C), 42.4 (d, J=14.0 Hz, CH₂), 124.0 (s, CH_{Ar}), 126.2 (s, CH_{Ar}), 133.1 (d, J=3.5 Hz, C_{Ar}), 140.0 (d, J=1.8 Hz, C_{Ar}), 145.7 (s, C_{Ar}), 147.0 (d, J=5.2 Hz, C_{Ar}). HRMS (EI): calculated m/z (C₃₁H₄₈N₁O₂P₁) 497.34172; found 497.34214.MS (EI, 70 eV): m/z (I, %): 497 (69), 482 (100), 439 (40), 57 (46). Anal. calculated for C₃₁H₄₈N₁O₂P₁: C, 74.81; H, 9.72; N, 2.81; P, 6.22. Found: C. 73.67; H, 9.65; N, 2,65; P, 6.56.

### Example 3

### N-(2,4,8,10-Tetra-tert-butyldibenz[d,f]{1,3,2}dioxaphosphepin-6-yl)diethylamine

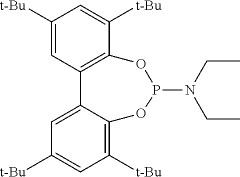 [SMILES: CCN(CC)p1oc2c(C(C)(C)C)cc(C(C)(C)C)cc2c2cc(C(C)(C)C)cc(C(C)(C)C)c2o1]

(1b)

The compound was prepared analogously to the method of Example 1. Yield: 51%; white solid (recrystallized from CH₃CN/toluene (10/1); ¹H NMR (300 MHz, CDCl₃): δ 0.94 (br, s, 6H), 1.27 (s, 18H), 1.40 (s, 18H), 2.90 (br, s, 4H), 7.08 (d, 2H, J=2.4 Hz), 7.32 (d, 2H, J=2.5 Hz). ³¹P NMR (121 MHz, CDCl₃): δ 148.4 (s). ¹³C NMR (75 MHz, CDCl₃): δ 15.6 (br, s, CH₂CH₃), 31.0 (d, J=2.8 Hz, (CH₃)₃C), 31.6 (s, (CH₃)₃C), 34.6 (s, (CH₃)₃C), 35.4 (s, (CH₃)₃C), 40.7 (br, s, CH₃CH₂), 123.9 (s, CH_{Ar}), 126.3 (s, CH_{Ar}), 132.5 (d, J=3.6 Hz, C_{Ar}), 139.7 (d, J=1.3 Hz, C_{Ar}), 145.4 (s, C_{Ar}), 147.8 (d, J=5.4 Hz, C_{Ar}). HRMS (ESI-TOF/MS): calculated m/z (C₃₂H₅₁N₁O₂P₁, (M+H)⁺) 512.36519; found 512.36531; calculated m/z (C₃₂H₅₀N₁Na₁O₂P₁, (M+Na)⁺) 534.34714; found 534.34781.MS (EI, 70 eV): m/z (I, %): 511 (62), 496 (35), 439 (100), 72 (28), 57 (39).

### Example 4

### N-(2,4,8,10-Tetra-tert-butyldibenz[d,f]{1,3,2}dioxaphosphepin-6-yl)-N-methylaniline

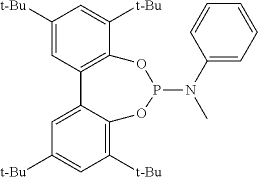 [SMILES: CN(c1ccccc1)p1oc2c(C(C)(C)C)cc(C(C)(C)C)cc2c2cc(C(C)(C)C)cc(C(C)(C)C)c2o1]

(1c)

The compound was prepared analogously to the method of Example 1. Yield: 34%; white solid (after recrystallizing twice from CH₃CN/toluene (3/2)); ¹H NMR (300 MHz, CDCl₃): δ 1.29 (s, 18H), 1.36 (s, 18H), 2.71 (s, 3H), 6.90 (t, 1H, J=7.2 Hz), 7.12 (d, 2H, J=2.4 Hz), 7.14-7.28 (m, 4H), 7.33 (d, 2H, J=2.4 Hz). ³¹P NMR (121 MHz, CDCl₃): δ 147.8 (br, s). ¹³C NMR (62 MHz, CDCl₃): δ 30.9 (d, J=2.9 Hz, (CH₃)₃C), 31.6 (s, (CH₃)₃C), 33.1 (br, s, NCH₃), 34.6 (s, (CH₃)₃C), 35.5 (s, (CH₃)₃C), 119.6 (d, J=16.5 Hz, CH_{Ar}), 122.0 (s, CH_{Ar}), 124.2 (s, CH_{Ar}), 126.5 (s, CH_{Ar}), 128.9 (s, CH_{Ar}), 132.4 (d, J=3.7 Hz, C_{Ar}), 139.9 (d, J=1.5 Hz, C_{Ar}), 146.1 (s, C_{Ar}), 146.7 (s, C_{Ar}), 147.5 (d, J=5.5 Hz, C_{Ar}).MS (EI, 70 eV): m/z (I, %): 545 (30), 439 (100), 57 (30). Anal. calculated for C₃₅H₄₈N₁O₂P₁: C, 77.03; H, 8.87; N, 2.57; P, 5.68. Found: C, 76.74; H, 9.05; N, 2.26; P, 5.76.

### Example 5

### N-(2,4,8,10-Tetra-tert-butyldibenz[d,f]{1,3,2}dioxaphosphepin-6-yl)piperidine

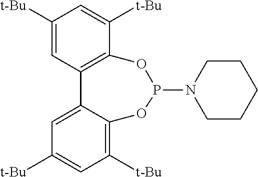 [SMILES: CC(C)(C)c1cc(C(C)(C)C)c2op(N3CCCCC3)oc3c(C(C)(C)C)cc(C(C)(C)C)cc3c2c1]

(1d)

The compound was prepared analogously to the method of Example 1. Yield: 92%; white solid. ¹H NMR (300 MHz, CDCl₃): δ 1.27 (s, 18H), 1.40 (s, 18H), 1.20-1.53 (m, 6H), 2.86 (br, s, 4H), 7.08 (d, 2H, J=2.5 Hz), 7.32 (d, 2H, J=2.5 Hz). ³¹P NMR (121 MHz, CDCl₃): δ 144.4 (s). ¹³C NMR (75 MHz, CDCl₃): δ 25.0 (s, CH₂), 27.4 (br, s, CH₂), 31.0 (d, J=2.7 Hz, (CH₃)₃C), 31.6 (s, (CH₃)₃C), 34.6 (s, (CH₃)₃C), 35.4 (s, (CH₃)₃C), 45.8 (br, s, CH₂), 124.0 (s, CH_{Ar}), 126.2 (s, CH_{Ar}), 132.7 (d, J=3.4 Hz, C_{Ar}), 139.8 (d, J=1.5 Hz, C_{Ar}), 145.5 (s, C_{Ar}), 147.5 (d, J=5.4 Hz, C_{Ar}). HRMS (ESI-TOF/MS): calculated m/z (C₃₃H₅₁N₁O₂P₁, (M+H)⁺) 524.36519; found 524.36557. MS (EI, 70 eV): m/z (I, %): 523 (28), 439 (12), 84 (6), 57 (12), 45 (100). Anal. calculated for C₃₃H₅₀N₁O₂P₁, C, 75.68; H, 9.62; N, 2.67; P, 5.91. Found: C, 75.85; H, 9.58; N, 2.78; P, 6.12.

### Example 6

General method for the synthesis of Rh(acac)(CO)L from the transition metal precursor. To a stirred solution of Rh(acac)(CO)₂ (1 mmol) in dried CH₂Cl₂ (8 ml) was added dropwise, within 40 min, a solution of the phosphoramidite (1 mmol) in dried CH₂Cl₂ (8 ml). The solution was stirred at room temperature for 2 h. Subsequently, the solvent was distilled off under reduced pressure and the residue was dried in vacuo for 1 h.

### Example 7

### Rh-Containing Complex with Ligand (1d)

The compound was synthesized analogously to the method detailed in Example 6. Yield: 96%; light grey powder. ¹H NMR (300 MHz, CDCl₃): δ 1.26-1.36 (m, overlapping signals, 3H), 1.27 (s, 18H), 1.36-1.44 (m, overlapping signals, 3H), 1.48 (s, 18H), 1.89 (s, 3H), 1.98 (s, 3H), 3.16 (br, s, 4H), 5.40 (s, 1H), 7.08 (d, 2H, J=2.4 Hz), 7.37 (d, 2H, J=2.4 Hz). ³¹P NMR (121 MHz, CDCl₃): δ 142.39 (d, ¹J_{RhP}=276.7 Hz). ¹³C NMR (75 MHz, CDCl₃): δ 24.8 (s, CH₂), 26.4 (d, J=3.2 Hz, CH₂), 27.1 (s, CH_{3acac}), 27.5 (d, J=7.9 Hz, CH_{3acac}), 31.4-31.5 (2× overlapping singlets, 2× (CH₃)₃C), 34.6 (s, (CH₃)₃C), 35.6 (s, (CH₃)₃C), 47.7 (s, CH₂), 100.6 (d, J=2.1 Hz, CH_{acac}), 124.6 (s, CH_{Ar}), 126.7 (s, CH_{Ar}), 131.6 (d, J=2.4 Hz, C_{Ar}), 140.2 (d, J=3.8 Hz, C_{Ar}), 146.6 (s, C_{Ar}), 146.7 (s, C_{Ar}), 185.3 (s, CH₃CO_{acac}), 187.4 (s, CH₃CO_{acac}). HRMS (ESI-TOF/MS): calculated m/z (C₃₉H₅₇N₁Na₁O₅P₁Rh₁, (M+Na)⁺) 776.29216; found 776,29243. MS (EI, 70 eV): m/z (I, %): 753 (19), 725 (100), 439 (13), 84 (23), 57 (33). IR (CaF₂ cuvette 0.1 mm, 0.1 M solution in toluene): 2005 cm⁻¹ (CO band).

### Example 8

In one embodiment of the invention, the hydroformylation was conducted in a 200 ml autoclave equipped with pressure-retaining valve, gas flow meter, sparging stirrer and pressure pipette as reaction zone. To minimize the influence of moisture and oxygen, the toluene used as solvent was treated with sodium ketyl and distilled under argon. The mixture of the n-octenes used as substrate was heated at reflux over sodium and distilled under argon for several hours. The transition metal was added as a precursor in the form of [(acac)Rh(COD)] (acac=acetylacetonate anion; COD=1,5-cyclooctadiene), dissolved in toluene. The latter was mixed with a solution of the respective ligand in the autoclave under an argon atmosphere. The reactor was heated up under synthesis gas pressure and the unsaturated compounds, especially the olefin, the mixture of olefins, were introduced by means of a pressure-resistant pipette once the reaction temperature had been attained. In further embodiments of the process according to the invention, the unsaturated compounds to be hydroformylated were introduced into the reaction zone prior to the addition of the hydrogen- and carbon monoxide-containing gas mixture. This applies especially to unsaturated compounds present in a liquid state at room temperature and standard pressure. In these cases, there is no need to add an external solvent, the solvents being the secondary products formed internally, for example those formed in situ during the reaction from the aldol condensation of the primary aldehyde product.

The reaction was conducted at constant pressure. After the reaction time had elapsed, the autoclave was cooled to room temperature, decompressed while stirring and purged with argon. 1 ml of each reaction mixture was removed immediately after the stirrer had been switched off, diluted with 5 ml of pentane and analyzed by gas chromatography. Inventive working examples are compiled in Tables 1 and 2, in which one entry also relates to the use of the phosphite ligands known by the CAS Registry Numbers [93347-72-9], [31570-04-4]—trade name Alkanox®240.

### Example 9

TABLE 1
Hydroformylation of 1-octene^{a}

| | Yield Of | | |
|---|---|---|---|
| Ligand | aldehydes [%] | n-Nonanal [%] | TOF_{40 min} [h⁻¹] |
| 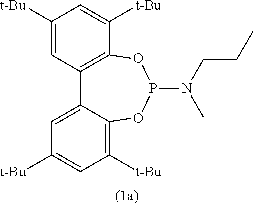 [SMILES: CCCN(C)p1oc2c(C(C)(C)C)cc(C(C)(C)C)cc2c2cc(C(C)(C)C)cc(C(C)(C)C)c2o1] | 98 | 55.5 | 8020 |
| 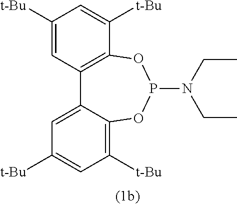 [SMILES: CCN(CC)p1oc2c(C(C)(C)C)cc(C(C)(C)C)cc2c2cc(C(C)(C)C)cc(C(C)(C)C)c2o1] | 97 | 55.0 | 7940 |

^{a}conditions: [Rh] = 0.01728 mmol; 40 ppm Rh; P/Rh = 5:1, 50 bar CO/H₂, [1-octene] = about 94 mmol; tolune, 100° C.; 40 min.

TABLE 2
Hydroformylation of a mixture of n-octenes^{a,b}

| | | | Yield | Selectivity |
|---|---|---|---|---|
| Ligand | Structure | k_{obs.} [min⁻¹] | [%] | [%] |
| (1a) | 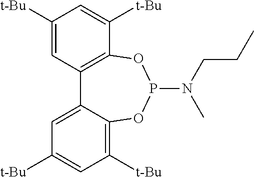 [SMILES: CCCN(C)p1oc2c(C(C)(C)C)cc(C(C)(C)C)cc2c2cc(C(C)(C)C)cc(C(C)(C)C)c2o1] | 0.199 | 99 | 17.5 |
| (1b) | 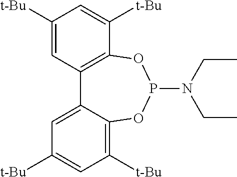 [SMILES: CCN(CC)p1oc2c(C(C)(C)C)cc(C(C)(C)C)cc2c2cc(C(C)(C)C)cc(C(C)(C)C)c2o1] | 0.347 | 97 | 18.0 |
| (1d) | 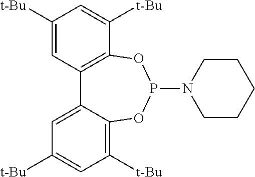 [SMILES: CC(C)(C)c1cc(C(C)(C)C)c2op(N3CCCCC3)oc3c(C(C)(C)C)cc(C(C)(C)C)cc3c2c1] | 0.198 | 98 | 16.0 |
| (1c) | 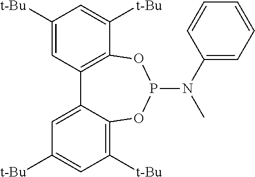 [SMILES: CN(c1ccccc1)p1oc2c(C(C)(C)C)cc(C(C)(C)C)cc2c2cc(C(C)(C)C)cc(C(C)(C)C)c2o1] | 0.099 | 96 | 23.8 |
| Comparative | Alkanox ® 240 as per CAS | 0.194 | 95 | 20.0 |
| ligand | Reg. No. [93347-72-9], | | | |
| | [31570-04-4] | | | |

^{a}for conditions see Table 1;
^{b}consisting of: 1-octene, 3%; cis + trans-2-octene, 49%; cis + trans-3-octene, 29%; cis + trans-4-octene, 16%; structurally isomeric octenes, ~3%.

The relative activities are determined by the ratio of 1st order k to k0, i.e. the k value at time 0 in the reaction (start of reaction), and describe the relative decrease in activity during the experiment duration.

The 1st order k values are obtained from a plot of (-In(1-conversion)) against time.

The hydroformylation results in Tables 1 and 2 reveal that the inventive phosphoramidites (1a) to (1d) have at least comparable results in terms of catalytic efficacy—measured as k_{obs}. [min⁻¹]—and in terms of yield and the n-selectivity with the comparative Alkanox®240 ligand as per CAS Reg. No. [93347-72-9], [31570-04-4], and are even superior to the comparative ligand in some of these individual features.

### Example 10

Hydrolysis Experiments.

To a 0.0175 M solution of the phosphoramidite in dried 1,4-dioxane were added 20 equivalents of distilled water. This sample was divided between two NMR tubes which had been dried under reduced pressure beforehand in a flame and which contained tri-n-octylphosphine oxide in o-xylene-D10 as external standard. For comparison, one sample was stored at room temperature, the other heated to 80-85° C. If the compound was stable over a prolonged period at this temperature, the temperature was increased to 100° C. The samples were analyzed quantitatively by means of ³¹P NMR (manually adjusted lock signal based on CDCl₃, NS=256, D1=5 sec).

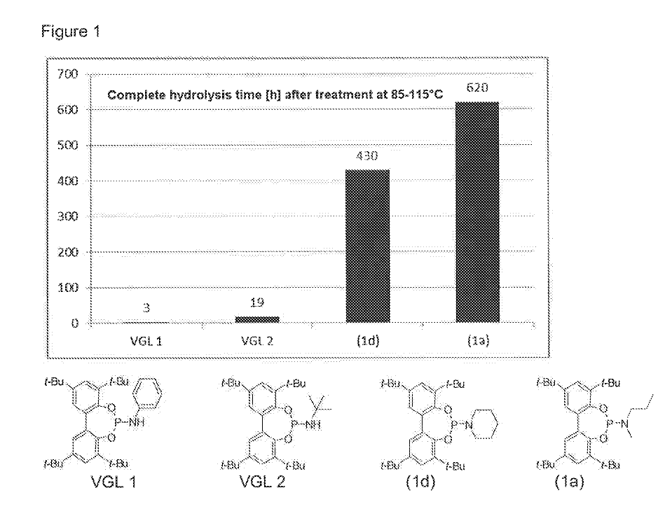

As is apparent from FIG. 1, the two phosphoramidites of the formulae (1a) and (1d), which derive from a secondary amine with low steric hindrance, are many times more stable than those phosphoramidites which derive from a primary amine (VGL 1 and VGL 2 each=comparative ligand).

The inventive ligands (1a) and (1d) thus achieve the stated object because of their exceptional hydrolysis stability, as already detailed above.

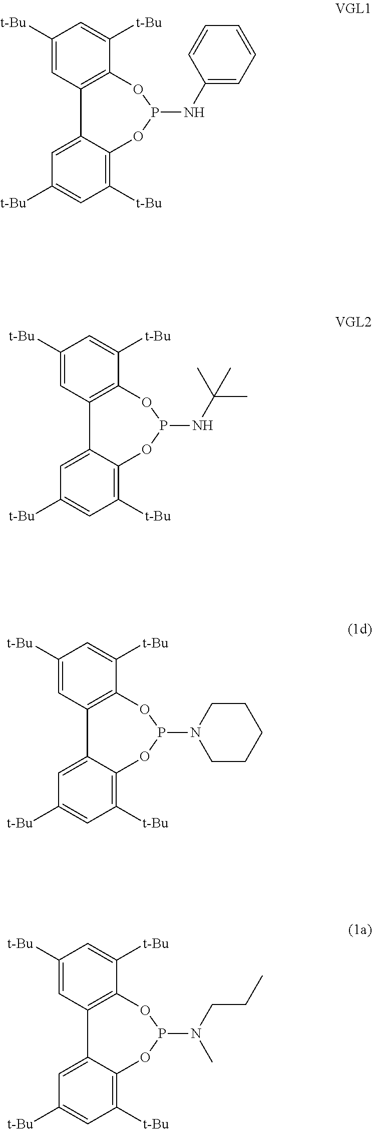 [SMILES: CC(C)(C)c1cc(C(C)(C)C)c2op(Nc3ccccc3)oc3c(C(C)(C)C)cc(C(C)(C)C)cc3c2c1 ; CC(C)(C)Np1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1 ; CCCN(C)p1oc2c(C(C)(C)C)cc(C(C)(C)C)cc2c2cc(C(C)(C)C)cc(C(C)(C)C)c2o1]

1. Phosphoramidites, of the formulae (I) 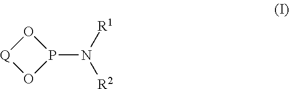
where Q is a divalent substituted or unsubstituted aromatic radical;
where R¹ is selected from alkyl, substituted or unsubstituted cycloalkyl and aryl radicals;
where R² is selected from alkyl, substituted or unsubstituted cycloalkyl and aryl radicals, but R¹ and R² are not i-propyl radicals, or R¹ and R² together with N form a heterocyclic structure.

2. Phosphoramidites according to claim 1, where Q is selected from substituted or unsubstituted 1,1′-biphenyl, 1,1′-binaphthyl and ortho-phenyl radicals.

3. Phosphoramidites according to claim 2, where Q is selected from substituted or unsubstituted 1,1′-biphenyl radicals.

4. Phosphoramidites according to claim 3, where R¹ is selected from C₁-C₅-alkyl, substituted or unsubstituted cycloalkyl and aryl radicals;
R² is selected from C₁-C₅-alkyl, substituted or unsubstituted cycloalkyl and aryl radicals, but R¹ and R² are not i-propyl radicals, or R¹ and R² together with N form a heterocyclic structure via alkylene groups.

5. Phosphoramidites according to claim 4, where R¹ is selected from C₁-C₅-alkyl, C₄-C₆-cycloalkyl and phenyl radicals; R² is selected from C₁-C₅-alkyl, C₄-C₆-cycloalkyl and phenyl radicals, but R¹ and R² are not i-propyl radicals, or R¹ and R² together with N form a heterocyclic structure via alkylene groups.

6. Phosphoramidites according to claim 5, where the compounds of the formula (I) are selected from:

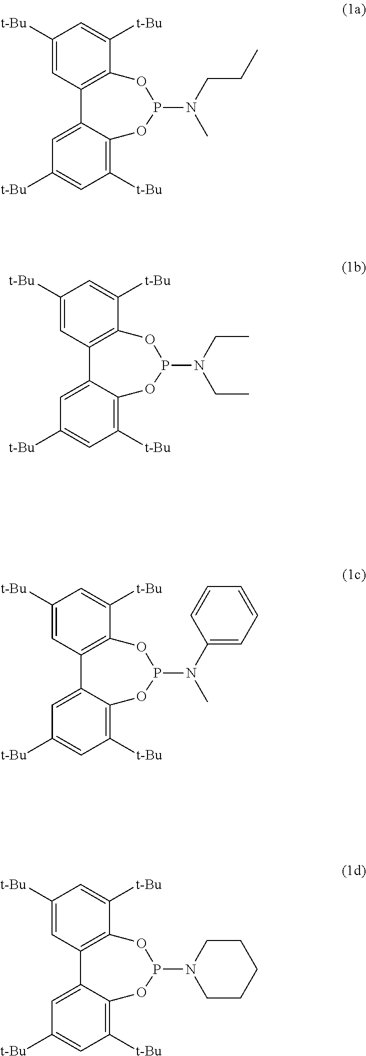 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

7. Transition metal compounds of the formula Me(acac)(CO)L with Me=transition metal, where L is selected from: 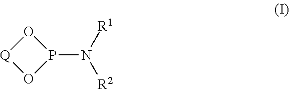
where Q is a divalent substituted or unsubstituted aromatic radical;
where R¹ is selected from alkyl, substituted or unsubstituted cycloalkyl and aryl radicals;
R² is selected from alkyl, substituted or unsubstituted cycloalkyl and aryl radicals, but R¹ and R² are not i-propyl radicals, or R¹ and R² together with N form a heterocyclic structure.

8. Transition metal compounds according to claim 7, where Q is selected from substituted or unsubstituted 1,1′-biphenyl, 1,1′-binaphthyl and ortho-phenyl radicals.

9. Transition metal compounds according to claim 8, where Q is selected from substituted or unsubstituted 1,1′-biphenyl radicals.

10. Transition metal compounds according to claim 9, where R¹ is selected from C₁-C₅-alkyl, substituted or unsubstituted cycloalkyl and aryl radicals;
R² is selected from C₁-C₅-alkyl, substituted or unsubstituted cycloalkyl and aryl radicals, but R¹ and R² are not i-propyl radicals, or R¹ and R² together with N form a heterocyclic structure via alkylene groups.

11. Transition metal compounds according to claim 10, where R¹ is selected from C₁-C₅-alkyl, C₄-C₆-cycloalkyl and phenyl radicals; R² is selected from C₁-C₅-alkyl, C₄-C₆-cycloalkyl and phenyl radicals, but R¹ and R² are not i-propyl radicals, or R¹ and R² together with N form a heterocyclic structure via alkylene groups.

12. Transition metal compounds of the formula Me(acac)(CO)L with Me=transition metal according to claim 11, where L is selected from:

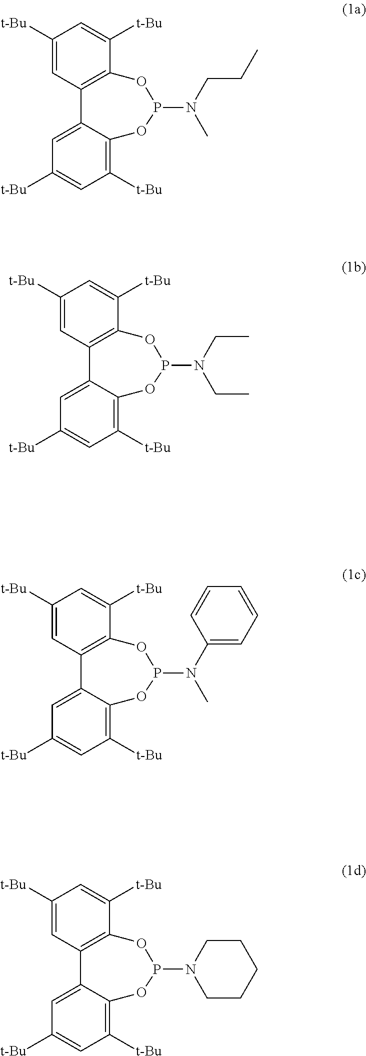 [SMILES: CCCN(C)p1oc2c(C(C)(C)C)cc(C(C)(C)C)cc2c2cc(C(C)(C)C)cc(C(C)(C)C)c2o1 ; CCN(CC)p1oc2c(C(C)(C)C)cc(C(C)(C)C)cc2c2cc(C(C)(C)C)cc(C(C)(C)C)c2o1 ; CN(c1ccccc1)p1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3CCCCC3)oc3c(C(C)(C)C)cc(C(C)(C)C)cc3c2c1]

13. Transition metal compounds of the formula Me(acac)(CO)L with Me=transition metal according to claim 12, where Me is selected from rhodium, iridium, ruthenium, cobalt.

14. Transition metal compounds according to claim 13, where the transition metal is rhodium.

15. Catalytically active compositions in the hydroformylation comprising:
a) transition metal compounds according to claims 7-14;
b) free ligands according to claims 1-6;
c) solvents.

16. Use of a catalytically active composition according to claim 15 in a process for hydroformylating unsaturated compounds.

17. Process for hydroformylating unsaturated compounds using a catalytically active composition according to claim 15, where the unsaturated compounds are selected from:
hydrocarbon mixtures from steamcracking plants;
hydrocarbon mixtures from catalytically operated cracking plants;
hydrocarbon mixtures from oligomerization processes;
hydrocarbon mixtures comprising polyunsaturated compounds;
olefin-containing mixtures including olefins having up to 30 carbon atoms.

18. Process according to claim 17, wherein, in a first process step, phosphoramidites according to claims 1-6 are initially charged as ligands in at least one reaction zone, and reacted with a precursor of the transition metal to give a transition metal compound according to claims 7-14 and finally, after adding free ligands according to claims 1-6, and also solvents and a carbon monoxide- and hydrogen-containing gas mixture, to give a catalytically active composition according to claim 15; in a subsequent step, the unsaturated compounds are added under the reaction conditions to form a polyphasic reaction mixture;
after the end of the reaction, the reaction mixture is separated into aldehydes, alcohols, high boilers, ligands, degradation products of the catalytically active composition.

19. Polyphasic reaction mixture comprising:
unsaturated compounds,
a gas mixture including carbon monoxide, hydrogen;
aldehydes,
catalytically active compositions according to claim 15.
